(19) United States
(12) Patent Application Publication
(10) Pub. No.: US 2024/0008897 A1
(43) Pub. Date: Jan. 11, 2024

(54) SURGICAL DEVICE, A SYSTEM, AND A METHOD OF MANUFACTURING A SURGICAL DEVICE

(71) Applicant: ZURIMED TECHNOLOGIES AG, Zürich (CH)

(72) Inventors: Elias Bachmann, Zürich (CH); Xiang Li, Zumikon (CH); Pol Banzet, Zürich (CH)

(21) Appl. No.: 18/252,653

(22) PCT Filed: Nov. 12, 2020
(86) PCT No.: PCT/EP2020/081887

§ 371 Date: May 11, 2023

(51) Int. Cl. A61B 17/34

(52) U.S. Cl. CPC A61B 17/3468

(57) ABSTRACT

The present disclosure shows a surgical device for felting an implant to soft tissue of a patient. The surgical device includes at least one felting needle. The at least one felting needle is configured to move reciprocally. Further, the surgical device includes a connection interface for connecting the at least one felting needle to an actuator and for transferring a reciprocal motion from the interface to the at least one felting needle. The device includes a needle protection mechanism. The needle protection mechanism prevents the at least one needle from being damaged during the reciprocal motion due to a contact with a rigid structure.

### CROSS-REFERENCE TO RELATED APPLICATION(S)

This Application is a Section 371 National Stage Entry Application of International Application No. PCT/EP2020/081887, filed Nov. 12, 2020 and published as WO 2022/100833 on May 19, 2022, in English.

### FIELD

The present invention relates to a surgical device, a system, and a method of manufacturing a surgical device according to the preambles of the independent claims.

### BACKGROUND

The present applicant recently developed a surgical felting device allowing a biomechanically advantageous implantation of implants. The developed device allows an improved fixation as compared to conventional suturing techniques. One example of such a surgical felting device is disclosed in PCT/CH2019/000015. The surgical felting device comprises a needle that repeatedly moves through a surgical felt and into tissue. By embedding strands of the felt inside the tissue, the needle creates a strong and well distributed mechanical bond between the felt and the tissue. Compared to conventional suturing, this technique is faster and alleviates adverse effects such as “cheesewiring” of suture, where the suture cuts through tissue which can happen through local stress peaks.

However, since the surgical felting device may be handheld and the needle moves at high speeds, there is high risk of needle damage. The damage may result from collisions between the needle and rigid structures such as bones or other surgical tools. Such collisions could lead to a partial destruction of the needle (plastic bending, breaking or splintering) which adversely influences the functionality. Even further, parts of the needle may be lost inside the patient's body, presenting a health risk. Lastly, the high speeds of the needles in the tissue and in the surgical felt may generate undesirable heat that results from friction.

Disclosed embodiments of the present invention help to overcome the above disadvantages of the prior art. In particular, disclosed embodiments of the present invention provide a surgical felting device that is safe to use for the operator and the patient. A further benefit of embodiments of the present invention may be to reduce the heat generated by the felting needle.

### SUMMARY

One aspect of the invention relates to a surgical device for felting an implant to soft tissue of a patient. The patient may be a human or an animal. Felting may be understood as the process of moving a felting needle repeatedly into, and in some embodiments through, an implant comprising a felt and thereby entangling fibers of a felt. A felting needle may be understood as a needle including barbs that catch the fibers of the felt and move the fibers through the felt and which entangles the fibers of the felt further. In particular, the felting may not only entangle the fibers of a felt that forms part of an implant with each other but also entangle the fibers of the tissue of a patient with the fibers of the felt. Thereby, some strands of the felt become embedded in and entangled with the patient's tissue and some strands of the tissue fibers may become embedded in and entangled with the felt. This creates a strong and well distributed mechanical bond between tissue and felt.

The surgical device comprises at least one felting needle. The at least one felting needle is configured to move reciprocally. A reciprocating motion as mentioned herein may be understood to be a repetitive back and forth motion. The motion may be translational, i.e. linear, or rotational. Further, the surgical device comprises a connection interface for connecting the at least one felting needle to an actuator and for transferring a reciprocal motion from the interface to the at least one felting needle. The interface may provide a releasable or a permanent attachment. For example, the releasable interface may include pins, latches, screws, magnets or other known mechanical or other connection means. A permanent attachment using rivets, glue or a one-piece design is also possible. A releasable attachment of the at least one needle would allow the at least one needle to be replaced.

In certain embodiments the device includes a water cooling system to cool down friction heat which is generated by the oscillating needle.

The actuator may be pneumatic, hydraulic or electromagnetic. In particular, the actuator may be an electric motor or may comprise or use electromagnetic coils or pneumatic or hydraulic pressure. The device may be configured to convert a rotational motion of the actuator to a translational motion. In particular, the device may comprise a scotch yoke mechanism or a piston rod drive mechanism to transform a rotational motion into a translational motion. Thereby, more efficient motors may be used. A speed and/or frequency of the reciprocal translational motion (oscillation) can be adjustable. The device may comprise a linear motion rod to transfer the reciprocal motion from the interface or the actuator to the at least one needle. To reduce wear and heating, bearings and low friction materials (e.g. polytetrafluorethylene) may be used to minimize friction of a linear motion rod.

The at least one needle is preferably made of or comprises stainless steel. The needle may include a stainless steel coating. Stainless steel is particularly resistant to the oxidizing conditions of the surgical operation. Hence, the present device may e.g. be used in arthroscopic surgeries, in which a constant saline flush is applied, that may oxidize the needle.

The device may comprise a needle protection mechanism. The needle protection mechanism prevents the at least one needle from being damaged during the reciprocal motion due to a contact with a rigid structure.

The needle protection mechanism may define a maximal penetration depth of the at least one needle. The maximal penetration depth may be adjustable. In particular, the maximum penetration depth may be less than the amplitude of the reciprocal motion. Additionally or alternatively, the needle protection mechanism may limit the reciprocal motion or may be configured to detect an obstacle (i.e. hard tissue) or may decouple an actuator from the at least one needle in case a collision occurs or any combination thereof. The motion may be limited by modifying the amplitude of the reciprocal motion and/or by limiting the maximal penetration depth.

The rigid structure may be for example the bone of a patient or another surgical tool that may come in contact the needle while the needle is actuated.

The surgical device may be used for the repair or fixation of anatomical structures such as: collagenous tissues such as tendons, menisici, spinal disks or fascia, ligament reconstructions (collateral ligaments, cruciate ligaments, etc.), subcutaneous sutures, conventional suturing of skin closures, skeletal muscle, heart muscle, valves, and hollow organs (large vessels, bladder, esophagus, possibly intestine). In addition, an implant may be attached to these anatomical structures.

In one embodiment, the at least one felting needle has a width or diameter of less than 0.8 mm, preferably less than 0.6 mm, most preferably less than 0.4 mm. Needles with a low diameter have a lower friction and thus less heat is generated. However, the lower needle diameters may lead to more fragile needles.

In one embodiment, the needle protection mechanism includes a spacer for contacting the soft tissue or patch such that the spacer sets a maximal predetermined penetration depth for the at least one needle. The spacer pushes against the tissue and thereby prevents the needle from penetrating further than desired. This may be especially useful for anatomical structures that are arranged close to bones such as tendons or spinal disks. Further, the spacer helps an operator to keep the amplitude of the motion of the needle within a desired range (e.g. the felt and the soft tissue) and improves handling.

The spacer may be elastic, in particular, the spacer may include or form a spring. Elastic may be understood as allowing the user to press the device against the soft tissue and thereby changing the spacing of the spacer dependent on the applied force. In one embodiment the spacer may have a curved or bent shape. The elasticity allows a constant contact of the tip of the device on the feltable patch or tissue. Further, this may help holding the soft tissue in place without the need for further tools and/or assistance. If there is no constant contact to the tissue or patch, the tissue or patch could vibrate and reduce a penetration of the needle through the tissue. In particular, the spacer prevents the needle from lifting the patch, when the needle is retracted (stripping effect). When the needle is retracted from the felt and/or the tissue, the barbs and/or friction may pull the patch towards the surgical device. The spacer prevents this and the at least one needle will pull out of the patch and/or the tissue without lifting it.

In a preferred embodiment, the spacer comprises one or more fingers. Preferably, the spacer comprises two, three or more fingers. The one or more fingers comprise a distal end surface for contacting the soft tissue such that the reciprocal motion of the needle does not exceed the predetermined penetration depth. The one or more fingers may be bent at their distal side such that a radial outer surface of the fingers forms the distal end surface(s) of the spacer. Thereby, the operator may visually observe the at least one felting needle allowing for a more precise control of the device. Further, the bent fingers may allow an elasticity of the fingers.

In some embodiments, the fingers may be connected to each other at their distal side, and in particular be formed by a single wire. The fingers may be made from a shape memory-alloy, e.g. nitinol. The fingers may have a compressed and expanded configuration. For example the fingers may be expelled from a distal end of a guide tube for the needle.

In a preferred embodiment, the spacer forms a slide such that the needle can be slid along a surface of the soft tissue. In particular, the one or more fingers may be bent in the same direction. The device may then be moved over the soft tissue in the direction in which the curvature points with ease while at the same time maintaining a constant penetration depth and applying a constant pressure keeping the soft tissue in place. The slide may alternatively be formed similarly to slides known from sewing machines.

In a preferred embodiment, the at least one felting needle has a maximal penetration depth. The maximal penetration depth may be adjustable. Thereby, the device may be adapted to the particular use case. Further, an operator may adjust the penetration depth in case the operation is close to hard tissue.

In a preferred embodiment, the reciprocal motion has an amplitude. The amplitude may be adjustable. Thereby, the device may be adapted to the particular use case. Further, an operator may adjust the maximal penetration depth by adjusting the amplitude in case the operation is (too) close to hard tissue.

In a preferred embodiment, the device comprises a guide tube. The guide tube surrounds the at least one needle and/or a translation rod at least partially. The translational rod may transfer the reciprocal motion from the interface to the at least one needle. The guide tube may be an outer tubular member. Further the guide tube may be connected to a spring in series or may be elastic.

In a preferred embodiment, the device may comprise a scale indicating the current penetration depth. In a preferred embodiment, the device may comprise a scale indicating the current maximal penetration depth.

In a preferred embodiment, the needle protection mechanism comprises a needle collision sensor. In a preferred embodiment, the collision sensor is configured to measure the distance to a rigid structure. The needle collision sensor may be a distance sensor, such as for example an ultrasonic sensor. The sensor may measure a distance between the device, in particular a housing or the guide tube, and a hard tissue. Thereby, hard tissue or other obstacles, that may damage the needle can be detected. Additionally, the device may comprise an indicator for indicating the sensed distance to a hard tissue or another obstacle to the operator. The indicator may be optical (i.e. a display or a diode) or haptical or acoustic.

In a preferred embodiment, the needle collision sensor is configured to measure a bending of the at least one needle and/or the linear motion rod. The sensor may be an elastic strain sensor and/or an electromagnetic sensor and/or piezoelectric sensor. When the at least one needle collides with hard tissue such as bone, the needle and/or linear motion rod may bend before breaking. The needle collision sensor detects the bending. This may cause a controller to decouple the needle from the actuator or changes to reduce the maximal penetration depth.

In a preferred embodiment, the surgical device comprises a controller. The controller is configured to receive a signal from the sensor and adjust the maximal penetration depth of the at least one needle based on the measurement of the needle collision sensor. Thereby an open control loop is provided allowing for a quick, automatic adjustment of the penetration depth in response to the detection of a collision or the detection of the threat of a collision. In some embodiments, the actuator may measure the current maximal penetration depth with a further sensor and the controller may be configured to receive a signal from the actuator for a closed loop control of the maximal penetration depth.

The surgical device may comprise an actuator for driving the reciprocal motion of the at least one needle. In a preferred embodiment, the surgical device comprises a coupling adapted to transfer a reciprocal motion from the actuator to the at least one needle. The coupling may be a slipper clutch. A slipper clutch may be understood herein as a clutch that decouples the needle from the actuator, when a threshold fore or threshold moment is exceeded. The slipper clutch may slip at least partially when a predetermined force or moment acting on the at least one needle is exceeded. The needle protection mechanism is thereby adapted to decouple the needle from the actuator to prevent the needle from being damaged when a collision occurs.

In a preferred embodiment, the slipper clutch includes at least one of: a pin and a chamfered face, a ball spring and a corresponding dent for receiving the ball and a spring bearing for the at least one needle.

In a preferred embodiment, the needle protection mechanism includes a predetermined breaking point. The predetermined breaking point breaks, if the force on the at least one needle exceeds a predetermined threshold. The predetermined threshold may be lower than the force necessary to break the needle.

In a preferred embodiment, the predetermined breaking point is part of a mechanical coupling for transferring forces onto the at least one needle. The mechanical coupling preferably includes a rod having the predetermined breaking point. In some embodiments, the predetermined breaking point may be provided by a structural weakening such as a tapering, or a ridge, an edge or a dent. In other embodiments, the predetermined breaking point is part of the at least one needle.

In a further embodiment, the device is configured to detect a broken needle. In particular the needle may be part of the electric circuit or may include a needle collision sensor as described above. Further the device may include an indicator, e.g. a warning light, that is configured to indicate optically, haptically or acoustically that the needle is broken.

A further aspect of the invention relates to a system comprising a surgical device as described above and an actuator. The actuator may be in particular an electric motor for driving the at least one needle with the reciprocal motion.

A further aspect of the invention relates to a method of manufacturing a surgical device. Preferably the surgical device is a surgical device as described above. The method includes the following steps:
Providing a surgical device with at least one felting needle, wherein the at least one felting needle is configured to move reciprocally and a connection interface for connecting the at least one felting needle to an actuator and for transferring a reciprocal motion from the interface for the actuator to the at least one needle,
Providing a needle protection mechanism, wherein the needle protection mechanism prevents the at least one needle from being damaged due to a contact with rigid structure during the reciprocal motion.

The present summary is provided only by way of example and not limitation. Other aspects of the present invention will be appreciated in view of the entirety of the present disclosure, including the entire text, claims, and accompanying figures.

### BRIEF DESCRIPTION OF THE DRAWINGS

Non-limiting embodiments of the invention are described, by way of example only, with respect to the accompanying drawings, in which:

FIG. 1: shows a schematic perspective view of a first embodiment of a surgical device according to the invention.

FIGS. 2A and 2B: show further details with regard to an adjustable maximal penetration depth of the surgical device according to FIG. 1.

FIGS. 3A and 3B: show alternative embodiments for a distal tip of the surgical device according to FIG. 1.

FIG. 4: shows a schematic perspective view of a second embodiment of a surgical device according to the invention.

FIG. 5: shows a cross-section of the surgical device according to FIG. 4.

FIGS. 6A and 6B: show an interface for the actuator of a surgical device according to the invention.

FIGS. 7A and 7B: show a further interface for the actuator of a surgical device according to the invention.

FIG. 8: shows a cross-section of a distal portion of a third embodiment of a surgical device according to the invention.

FIG. 9: shows a cross-section of a distal portion of a fourth embodiment of a surgical device according to the invention in a first position.

FIG. 10: shows a cross-section of a distal portion of the surgical device of FIG. 9 in a second position.

FIG. 11: shows a cross-section of a distal portion of a fifth embodiment of a surgical device according to the invention.

While the above-identified figures set forth one or more embodiments of the present invention, other embodiments are also contemplated, as noted in the discussion. In all cases, this disclosure presents the invention by way of representation and not limitation. It should be understood that numerous other modifications and embodiments can be devised by those skilled in the art, which fall within the scope and spirit of the principles of the invention. The figures may not be drawn to scale, and applications and embodiments of the present invention may include features, steps, and/or components not specifically shown in the drawings.

### DETAILED DESCRIPTION OF ILLUSTRATIVE EMBODIMENTS

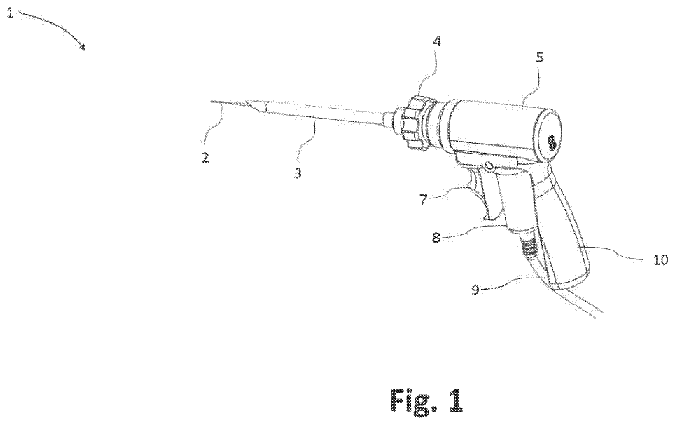

FIG. 1 shows a perspective view of a first embodiment of a surgical device 1 according to the invention. The surgical device 1 includes a housing 5 and a guide tube 3 extending from the housing 5. A felting needle 2 is arranged within the guide tube 5 and extends from the end of the guide tube of 5. The felting needle 2 can be moved reciprocally forwards and backwards along its axis and the axis of the guide tube 3. The surgical device 1 includes a handle 10 with a grip portion 7. Further, the surgical device 1 is connected to an energy source with a power cord 9 that connects to the surgical device.

An actuator—here electrical motor 8—is arranged within the housing 5 of the surgical device 1. The motor 8 drives the reciprocal motion of the needle 2. The guide tube 3 may be moved with respect to the needle 3 using wheel 4. When the wheel 4 is turned, the guide tube 3 is retracted in a distal direction such that the tip of the needle 2 is exposed. When the wheel 4 is turned in an opposite direction, the guide tube 3 may be pushed in a proximal direction such that the tip of the needle 2 can be covered.

Consequently, a penetration depth of the needle of the reciprocal motion of the needle is dependent on the relative position of the guide tube. If, for example, the needle 2 has a movement amplitude of 25 mm but the guide tube is only retracted by 10 mm from the most distal end position of the needle during the reciprocal motion, then the maximal penetration depth of the needle is only 10 mm. Proximal and distal are understood herein from the perspective of an operator, i.e. distal denotes a direction away from the operator and proximal a direction towards the operator.

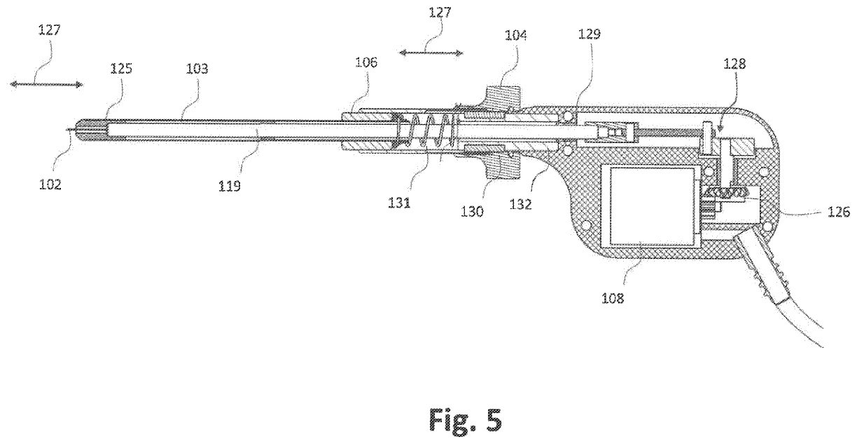

Setting the guide tube 3 relatively to the needle 2 limits the maximal penetration depth of the needle and allows an operator to avoid felting tissues that are deep below the implantation site and in particular to avoid collisions with hard tissues such as bone. Further, the guide tube 5 protects the needle 2 during transportation and unpacking of the device. The guide tube 5 is embedded in a guide plug 6 (see FIG. 1) and may be connected in series to a spring (see e.g. FIG. 5).

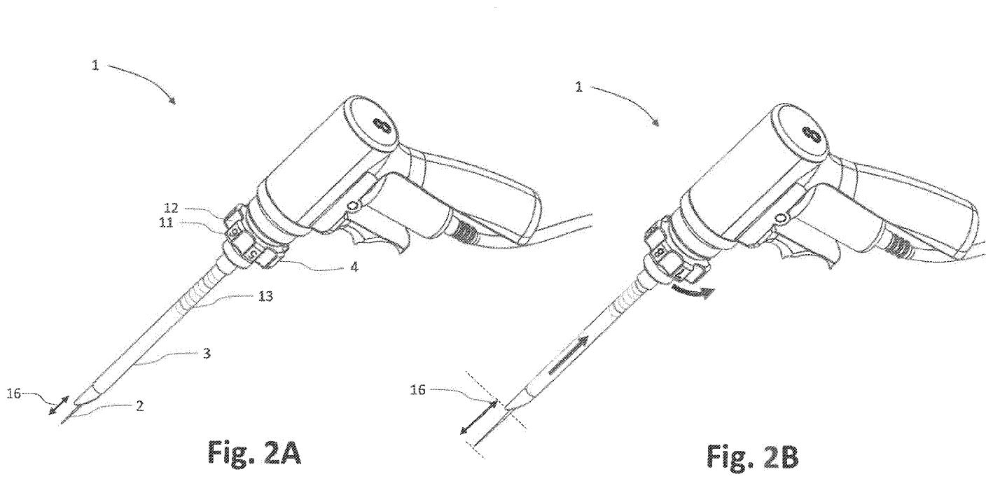

FIG. 2A shows the relative movement of the guide tube 3 in further detail. The wheel 4 includes ridges 12 such that an operator can grip the wheel more easily. Further, the wheel includes indicators 11 that show the current position of the guide tube 3. In the shown example, the number on the wheel indicates a length of the needle that can maximally be exposed, i.e. a maximal penetration depth. Further, the guide tube 3 itself includes a penetration depth scale 13. The penetration depth scale 13 is realized as markings on the outer surface of the guide tube 3. In the present example, the markings are circular rings around the guide tube 3. When the guide tube 3 is retracted, the current penetration depth can be read from the last ring that is still visible, i.e. not retracted into the housing 5. The depth scale shows the current penetration depth. In case the guide tube 3 is directly retracted as a result of the movement of the wheel 4, the current and maximal penetration depth are the same. However, in some embodiments these two may differ as will be explained with reference to the second embodiment.

The relative movement of the guide tube 3 is illustrated by the arrows in FIGS. 2A and 2B. If the operator turns the wheel counterclockwise, the guide tube 3 is retracted, exposing the indicated maximal penetration depth 16.

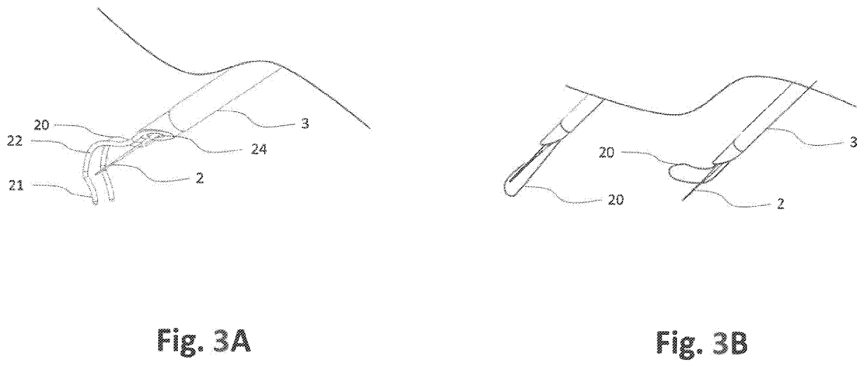

FIGS. 3A and 3B show further embodiments of the guide tube 3 that may be used independently or in combination with the retractable guide tube 3. In the embodiment of FIG. 3A, the guide tube 3 comprises at its distal end 24 a protective fork 20 with two fingers 21. The fingers 21 extend from the distal end and are bent around a curve 22. During use, the distal end surfaces of the fingers 21 are brought in contact with a soft tissue of a patient and define the penetration depth 16 similarly to the end of the guide tube 3 in the previously shown embodiment. The curvature of the fingers 21 allows the surgical device to slide smoothly over the soft tissue by helping the surgical device t to glide along the felt and/or soft tissue that are currently felted.

In the embodiment of FIG. 3B, the protective fork is formed by a wire made of a shape memory alloy. During transportation, the wire is held within the guide tube. Prior to an operation, the wire is expelled from the distal end 24 of the guide tube and assumes a bent position as shown on the right side in FIG. 3B. In case an operator pushes in a distal direction, the bends wire or the bent fingers may act as a spring allowing to temporarily (depending on the force) increase the penetration depth if necessary.

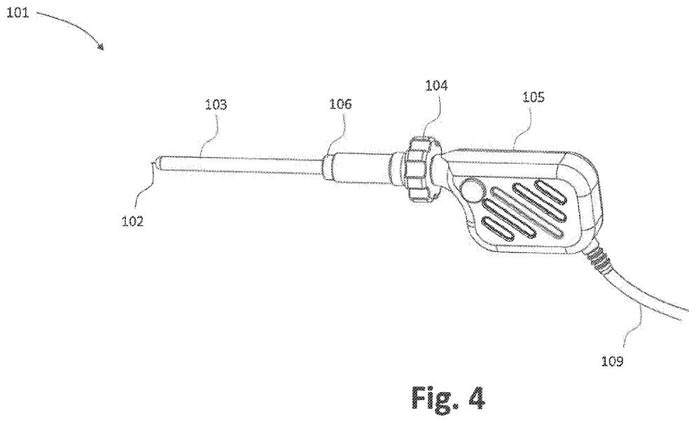

FIGS. 4 and 5 show a second embodiment of a surgical device 101 according to the invention. A perspective view of the surgical device 101 is shown in FIGS. 4 and a cross-section of the surgical device 101 is shown in FIG. 5. In general, the present description uses similar reference numerals for similar features, when the reference numeral increases by 100. For example, the felting needle 2 may be similar to the felting needle 102 of the embodiment shown in FIG. 4.

Similarly to the previously shown first embodiment, the second embodiment of a surgical device 101 includes a felting needle 102, a guide tube 103, a plug 106, a wheel 104 for retracting the guide tube 103, and a housing 105. A power cord 109 is connected to the housing 105.

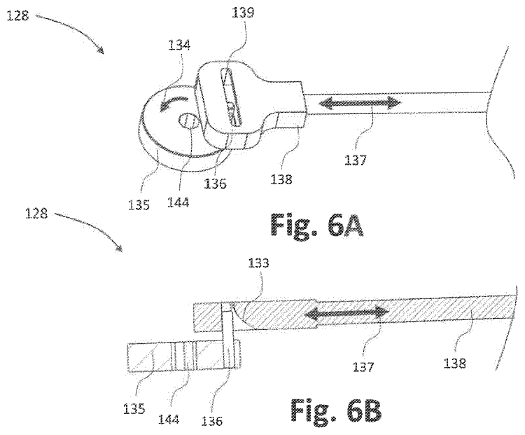

As can be seen from FIG. 5, the surgical device 101 includes a motor 108. The motor 108 drives an axis whose rotation is then transferred via gear units 126 to a scotch yoke 128. The scotch yoke 128 transforms the rotational motion of the motor 108 into a translational motion. One embodiment of a scotch yoke 128 can be seen in detail in FIGS. 6A and 6B. The scotch yoke 128 transfers the rational movement to a linear movement and the linear movement to translational motion rod 119 that is held within the guide tube 103. The translational motion rod 119 is guided by a bearing 129. The needle 102 is arranged at the distal end of the translational rod 119 and travels back and forth, wherein the amplitude of the movement is defined by the scotch yoke 128 and the frequency of the reciprocal movement is determined by the motor 108.

The surgical device 101 also includes a wheel 104. The wheel 104 includes an inner thread 130 that engages an outer threading of an insert 132. The outer tube 103 covers the translational rod 119 and is covered at its distal end with a distal tip 125. The proximal side and proximal end of the guide tube 3 includes a plug 106. The plug 106 is also connected to a spring 131 along its axial direction. Thereby the spring 131 is arranged between the plug 106 and the insert 132. During operation of the surgical device 101, the operator may push the distal tip with felting needle 102 onto a felt.

Thereby, the outer tube 103 is pushed in a proximal direction. The spring 131 resists this movement such that an operator has to push against it. Thereby, the outer tube 103 covers the sharp needle 102, when the device is not in use.

Further, the wheel 104 can be used to move the insert 132 back and forth. The insert delimits a maximum width 127 for the retraction of the outer tube 103. The maximum width 127 for the retraction thus corresponds to a maximal penetration depth of the needle 102. The delimitation of the maximal penetration depth protects the needle 102 from colliding with hard tissue, since the heart tissue (e.g. bony tissue) may be arranged below the soft tissue. Setting the maximal penetration depth as described is advantageous, if different kinds of tissues with hard tissue beneath are felted (e.g. an 8 mm rotator cuff tendon or a 12 mm Achilles tendon).

Besides the protection function, the spring 131 may also allow a constant contact of the tip of the device on the feltable patch, respectively tissue. If there is no constant contact to the tissue or felt, the tissue could vibrate and minimize penetration of the needle through the tissue.

FIGS. 6A and 6B show an embodiment of the scotch yoke 128 in detail. The scotch yoke may form an interface or a coupling. The scotch yoke 128 includes a wheel 135. The wheel 135 is driven by the motor 108 through the hexagonal socket 144 and rotates around direction 134. On a radially outer part of the wheel, a pin 136 is arranged. Further, the scotch yoke includes a slider 138. The slider 138 includes a slot 139, in which the pin 136 travels. Due to the rotation of the wheel forces in a linear direction along the axis of translational rod 119 are transferred as indicated by arrows 137 while the forces in the direction transversal thereto are not transferred due to the slot 139. FIG. 6B shows an embodiment that shows an example of a mechanical uncoupling between the needle 102 and the motor 108. The slider 138 may include a chamfered face 133 at the slot 139. If excessive forces are applied onto the needle 102, these forces are transferred via the linear motion rod and the slider to the scotch yoke 128. The chamfered face 133 glides onto the pin and thereby lifts the slider 137 out of the pin 136 preventing the application of further forces onto the needle 102. Hence, the wheel 135 and the slider 138 are mechanically decoupled.

In a further example, an electromagnetic motor 108 may be used. In this case, the amplitude of the needle 102 may be adjusted, if the motor detects forces above a threshold on the needle 102. If the forces on needle 102 exceed the electromagnetic forces of the linear drive, the needle may be retracted or, the motor may simply be stopped.

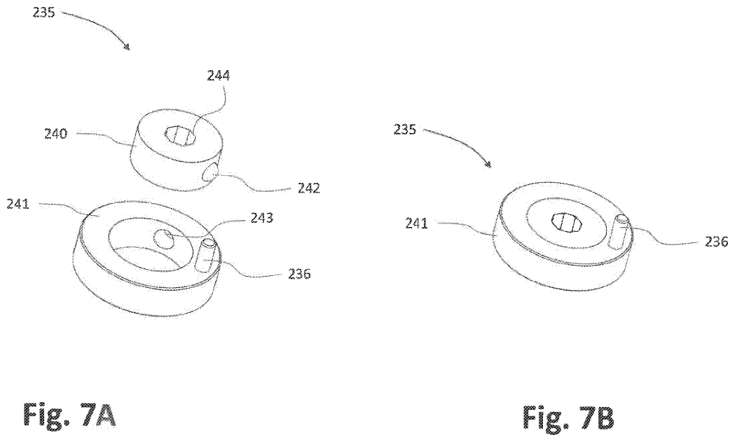

A further mechanical uncoupling is shown in FIGS. 7A and 7B. FIGS. 7A and 7B show a wheel 235. Wheel 235 is an alternative embodiment of the wheel 135 of FIGS. 6A and 6A. The wheel 235 includes two parts, an inner wheel 240 and a concentric outer wheel 241. FIG. 7A shows an exploded view of the wheel 235 and FIG. 7B shows the wheel 235 in an assembled form. Similarly to the wheel 135, the wheel 235 includes a hexagonal socket 244 and a pin 236. The inner wheel 240 is coupled to the outer wheel with a ball 242. The ball 242 is pushed radially outwardly by a spring (not shown). The outer wheel 241 includes a dent 243 along its inner circumference for the ball 242. When the inner wheel 240 is set into the inner circumference of the outer wheel 241, the ball 242 is pushed radially inwardly against the spring force and latches into the dent 243, if correctly aligned. As long as the ball 242 is in the dent 243, forces are transmitted from the motor 108 to the needle 102. However, in case the needle 102 collides with hard tissue, ball 242 is pushed inwards and no force beyond a threshold are transmitted. Thereby, the two wheels 240 and 241 are rotationally uncoupled.

Alternatively, the ball spring and dent may be replaced by a weak link that would break in case of excessive forces or a latch. A latch could be relying on springs or another compliant mechanism. A further alternative are magnets that may be finely tuned to disengage when needed and re-engage it when the force drops below a threshold. In other embodiments, the system can be electromechanical, with force sensors or other sensors (e.g. strain of the needle, conductivity) detecting that the forces on the needle exceed a threshold. A signal of these sensors may cause a controller to uncouple the needle from the actuator or stop the actuator.

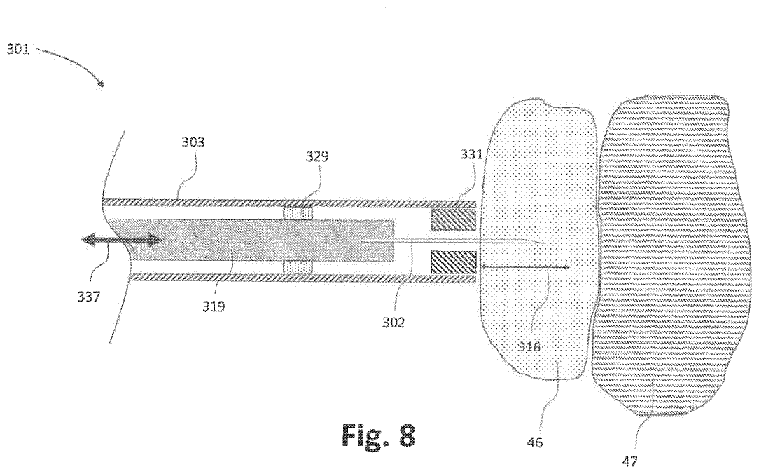

A third embodiment of a surgical device 301 according to the invention is shown in FIG. 8. The surgical device 301 includes a guide tube 303, and a needle 302 with a maximal penetration depth 316. As can be seen from FIG. 8, the needle is moved back and forth within soft-tissue 46. The needle 302 is driven by a translational rod 319 with a linear motion 337. The translational rod 319 is guided by bearing 329. Additionally, the surgical device 301 includes an ultrasonic distance sensor 331. The ultrasonic distance sensor 331 is arranged at the distal end of the guide tube and may measure a distance between a distal end of the guide tube 303 and bony tissue 47. The measured distance may be reported to a user with acoustic, optical or vibrational cues that allow the user to set the maximal penetration depth 316. For example, the maximal penetration depth may be set as described with respect to FIGS. 1 to 5. Alternatively, the surgical device 301 may additionally include a controller, that automatically adjusts the maximal penetration depth 316 to be less than the measured distance.

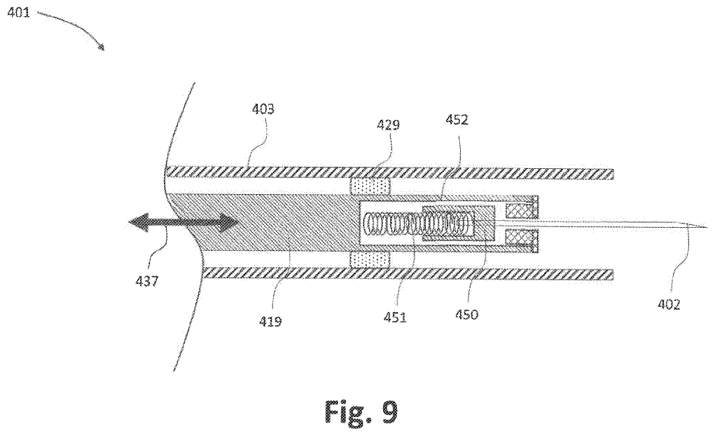

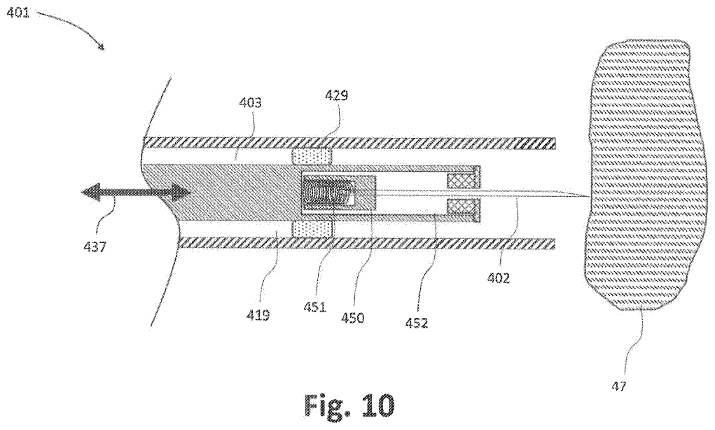

A fourth embodiment of a surgical device 401 is shown in FIGS. 9 and 10. The surgical device 401 is similar to the surgical device 301 and includes a guide tube 403, a bearing 429, and a translational rod 419 that moves along linear motion 437. The translational rod 419 is connected to the needle 402. However, the needle 402 is not directly connected to the translational rod 419. Instead, the translational forces of the translational rod 419 and transferred via a spring 451 and piston cylinder 450 onto the needle 402. The spring 451 and the cylinder 450 are arranged within a cavity 452 of the translational rod 419. Alternatively, the spring 451 and the cylinder 450 may be simply arranged at the end of the translational rod 419. During normal operation the needle 402 follows the movement of the translational rod 419. However, in case the needle 402 collides with a hard object, the spring 451 is compressed and absorbs the excessive forces (see FIG. 10). Additionally, a damping element may be included in parallel or series with the spring in order to prevent unwanted oscillations of the spring 451. In some embodiments, the spring may be formed by an elastically deformable rod (e.g. a nitinol band).

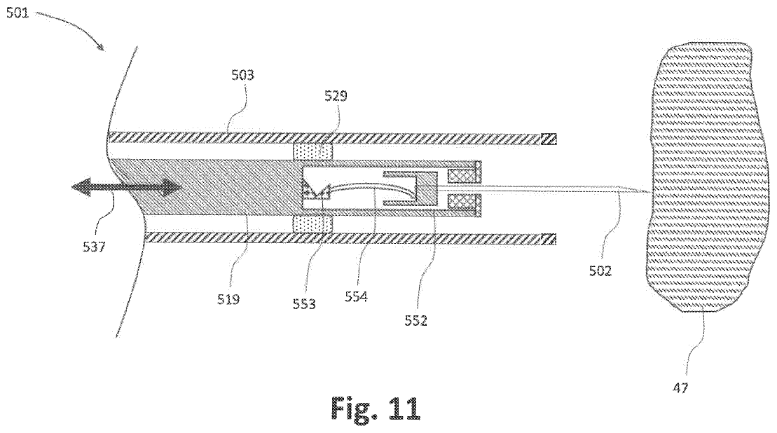

A fifth embodiment of a surgical device 501 is shown in FIG. 11. The surgical device 501 may be similar to the surgical device 401 shown in FIGS. 9 and 10. However, instead of a spring, the translational rod 519 is connected to the needle 502 with a connection rod 554 and a predetermined breaking point 553. The connection rod 552 may be an elastically deformable rod that is coupled to an element featuring a predetermined breaking point 553. The predetermined breaking point 553 may be formed by means known in the art e.g. by using edges, ridges or different materials. If the needle 502 collides with bony tissue 47, the connection rod 554 is deflected. Due to the deflection, translational forces are converted to shear forces on the predetermined breaking point 553. The higher the deflection of the rod, the higher the shear forces on the predetermined breaking point 553, which is consequently more likely to break.

Although the present invention has been described with reference to preferred embodiments, workers skilled in the art will recognize that changes may be made in form and detail without departing from the spirit and scope of the invention.

1. A surgical device for felting an implant to soft tissue of a patient comprising:
at least one felting needle configured to move reciprocally:
a connection interface for connecting the at least one felting needle to an actuator and for transferring a reciprocal motion from the interface to the at least one felting needle; and
a needle protection mechanism, wherein the needle protection mechanism is configured to prevent the at least one needle from being damaged due to a contact with rigid structure during the reciprocal motion.

2. The surgical device according to claim 1, wherein the at least one felting needle has a width or diameter of less than 0.8 mm.

3. The surgical device according to claim 1, wherein the needle protection mechanism includes a spacer for contacting the tissue such that the spacer sets a predetermined penetration depth for the at least one needle.

4. The surgical device according to claim 3, wherein the spacer comprises one or more fingers, wherein the one or more fingers comprise a distal end surface for contacting the tissue such that the reciprocal motion of the needle does not exceed the predetermined penetration depth.

5. (canceled)

6. The surgical device according to claim 1, wherein the at least one felting needle has a maximal penetration depth and wherein the maximal penetration depth is adjustable.

7. The surgical device according to claim 1, wherein the needle protection mechanism comprises a needle collision sensor.

8. The surgical device according to claim 7, wherein the needle collision sensor is configured to measure a bending of the at least needle.

9. The surgical device according to claim 7, wherein the surgical device comprises a controller, wherein the controller is configured to adjust a penetration depth of the at least one felting needle based on measurement of the needle collision sensor.

10. (canceled)

11. The surgical device according to claim 1, wherein the needle protection mechanism includes a slipper clutch, wherein the slipper clutch slips at least partially when a predetermined force or moment acting on the at least one needle is exceeded.

12. The surgical device according to claim 1, wherein the needle protection mechanism includes a slipper clutch, and wherein the slipper clutch includes at least one of: a pin and a chamfered face; a ball spring and a corresponding dent for receiving the ball; and/or a spring bearing for the at least one needle.

13. The surgical device according to claim 1, wherein the needle protection mechanism includes a predetermined breaking point wherein the predetermined breaking point breaks, if a force on the at least one needle exceeds a predetermined threshold.

14-15. (canceled)
